(19) United States
(12) Patent Application Publication
(10) Pub. No.: US 2023/0149633 A1
(43) Pub. Date: May 18, 2023

(54) ELECTROMECHANICAL CAP REMOVER

(71) Applicant: SHL MEDICAL AG, ZUG (CH)

(72) Inventor: Antonio Farieta, Baar (CH)

(21) Appl. No.: 17/915,078

(22) PCT Filed: May 7, 2021
(86) PCT No.: PCT/EP2021/062128

§ 371 Date: Sep. 27, 2022

(30) Foreign Application Priority Data
May 28, 2020 (WO) 20177055.9

(51) Int. Cl. A61M 5/32; A61M 5/50

(52) U.S. Cl. CPC A61M 5/3204; A61M 5/5086; A61M 2205/14; A61M 2205/276; A61M 2205/6072; A61M 2205/6081

(57) ABSTRACT

An electromechanical cap remover for removing a protective cap from a medicament delivery device is presented having a cap remover housing having an opening configured to receive the protective cap removably attached to the medicament delivery device, a motor system, a gripping device configured to be actuated by the motor system and to grip the protective cap when received in the opening a sensor unit configured to detect that the protective cap and a portion of a housing of the medicament delivery device is placed in the opening, a control unit configured to receive detection information from the sensor unit that the protective cap and the portion of the housing have been detected, wherein the control unit is configured to control the motor system to actuate the gripping device to grip the protective cap based on the detection information.

### TECHNICAL FIELD

The present disclosure generally relates to medicament delivery devices, and in particular to cap removers for medicament delivery devices.

### BACKGROUND

Medicament delivery devices comprise a delivery member for expulsion of a medicament. The delivery member may for example be a needle. The delivery member is typically shielded before administration to keep the delivery member sterile and to ensure user-protection from exposure to the delivery member. The medicament delivery device may therefore be provided with a flexible or rigid needle shield onto which a cap is fitted. The cap is removed before the medicament delivery devices is to be used, usually together with the flexible or rigid needle shield.

Often, the medicament administration is performed by the patient. Some patients may have difficulties in removing the cap, especially if they have impaired finger skills. A cap remover can therefore be used to facilitate the removal of the cap. US2018/0344947 A1 discloses a medical injector cap remover. The cap remover has a cap grabbing portion that has an internal opening which includes an extraction member having a series of laminas extending radially inwards.

One disadvantage with this cap remover is that it is only compatible with a limited number of medical injectors.

### SUMMARY

An object of the present disclosure is to provide an electromechanical cap remover which solves, or at least mitigates problems of the prior art.

There is hence provided an electromechanical cap remover for removing a protective cap from a medicament delivery device, the electromechanical cap remover comprising: a cap remover housing having an opening configured to receive the protective cap removably attached to the medicament delivery device, a motor system, a gripping device configured to be actuated by the motor system and to grip the protective cap when received in the opening, a sensor unit configured to detect that the protective cap and a portion of a housing of the medicament delivery device is placed in the opening, a control unit configured to receive detection information from the sensor unit that the protective cap and the portion of the housing have been detected, wherein the control unit is configured to control the motor system to actuate the gripping device to grip the protective cap based on the detection information.

The motor system thus actuates or drives the gripping device to grip the protective cap based on that it has been detected that the protective cap is in the opening. The gripping device may be able to grip different types of protective caps of different models of medicament delivery devices. For example, some protective caps may be gripped with more force than others, depending on their shape and size. Thus, the electromechanical cap remover may be more flexible than the cap remover disclosed in US2018/0344947 A1.

The actuation of the gripping devices involves moving the gripping device towards a central longitudinal axis of the opening so that it grips the protective cap.

The opening forms a cavity or channel extending inside the electromechanical cap remover.

The motor system may comprise one or more motors. The number of motors may depend on the number of degrees of freedom involved in actuating the gripping device.

The control unit may be configured to control the motor system to actuate the gripping device to grip the protective cap in response to receiving the detection information. Thus, in this case the term “based on” means “in response to receiving” the detection information.

According to one embodiment the sensor unit is configured to detect that the cap is maintained in the opening and that the portion of the housing is removed from the opening.

The control unit may be configured to control the motor to actuate the gripping device to release the protective cap in response to the control unit detecting that the housing has been removed. The actuation of the gripping devices involves moving the gripping device in a direction away from the central longitudinal axis of the opening so that it releases the protective cap.

According to one embodiment the sensor unit is configured to detect that the protective cap is removed from the medicament delivery device.

According to one embodiment the sensor unit is configured to read data or information from the protective cap and/or from any component of the medicament delivery device.

According to one embodiment the sensor unit is configured to read a barcode, QR code, and/or a colour code and/or an RFID tag or chip to obtain the data or the information. The sensor unit may thus comprise a barcode reader, a QR code reader, and/or an RFID tag or chip reader.

The senor unit may thus comprise a barcode reader, a QR code reader, and/or an RFID tag or chip and/or a colour sensor optionally with a light source.

According to one embodiment the sensor unit is configured to capture colours to obtain the data or the information. The sensor unit may thus comprise a colour sensor and optionally a light source.

One embodiment comprises a communication module configured to receive the data or the information from the sensor unit and to send it to an external device.

The communication module may be configured to communicate wirelessly or by wire with the external device. The communication module may thus be a communication module for wireless or wired communication.

The external device may for example be a smartphone, a tablet computer, a handheld computer, a server, or the cloud.

The communication module may be configured for two-way communication. The communication module may be configured to receive feedback data from the external device for example in response to sending the data or information to the external device. The feedback data may be based on the data or information.

According to one embodiment the data or the information comprises data or information related to the medicament delivery device and/or to the medicament.

The sensor unit may according to one example be configured to detect the temperature of the protective cap and the housing portion in the opening and provide the measured temperature as temperature data to the communication module. The communication module may be configured to send the temperature data to the external device.

The sensor unit may according to one example be configured to detect whether a breach in the storing temperature has previously occurred, for example if the cooling chain has been interrupted and the medicament delivery device reached a temperature above a certain temperature threshold. The communication module may be configured to send the temperature data to the external device.

The sensor unit may be configured to read a non-reversible temperature label provided on the protective cap or housing. The non-reversible temperature label may register that the temperature has reached a predetermined temperature, such as a temperature above an upper threshold temperature. The sensor unit may thereby be configured to detect whether a breach in storage temperature of the medicament delivery device and thus the medicament contained therein has occurred.

The sensor unit may according to one example be configured to detect whether the protective cap is properly attached to the housing or not and/or to detect whether the protective cap and/or housing portion has any damage. This may indicate a breach of sterility. The sensor unit may thus provide tampering or sterility breach data to the communication module. The communication module may be configured to send the tampering or sterility breach data to the external device.

According to one example, the communication module may receive feedback data from the external device based on the temperature data and/or the tampering or sterility breach data sent to the external device, wherein the control unit is configured to control the motor system to actuate the gripping device to grip the protective cap further based on the feedback data. For example, the control unit may be configured to be prevented from controlling the motor system to actuate the gripping device and grip the protective cap in case the feedback data contains such instructions even though the sensor unit has detected the presence of the protective cap. This may for example be the case if the temperature data indicates that the temperature measured by the sensor unit is higher than an upper threshold allowed or recommended for the medicament or that the medicament delivery device may be unsafe to use due to a breach of sterility. In this case, the control unit may be configured to provide feedback to the user based on the feedback data instead of controlling the motor. The electromechanical cap remover may for example comprise an indicator and the control unit may be configured to control the indicator to provide an indication to the user in case the feedback data comprises instructions preventing the control unit to control the motor system to actuate the gripping device to grip the protective cap. The indicator may be a visual indicator such as a display, an aural or tactile indicator, or a combination of two or more of these.

The data or information may for example comprise a unique identifier of the medicament delivery device and/or data or information identifying the medicament contained in the medical delivery device.

According to one embodiment the communication module is configured to receive feedback data from the external device, which feedback data is based on the data or information sent to the external device, wherein the control unit is configured to control the motor system to actuate the gripping device further based on the feedback data.

The control unit may be configured to control the motor system to actuate the gripping device in response to receiving the detection information and further based on the feedback data, the feedback data either confirming that the control unit is to control the motor system to actuate the gripping device or comprising instructions which prevents the control unit from controlling the motor to actuate the gripping device.

The data or information may according to one example include a best before date or production date of the medicament.

The communication module may for example receive feedback data from the external device based on the unique identifier of the medicament delivery device and/or the information identifying the medicament that the medicament delivery device has already been used previously because it has previously been read and/or that the medicament is currently not the correct medicament to be delivered and/or that the medicament has expired. In this case, the control unit may be configured to provide feedback to the user based on the feedback data instead of controlling the motor system. The control unit may be configured to control the indicator to provide an indication to the user based on the feedback data. This feedback may for example be that the medicament delivery device placed in the opening is not the correct one, that it has already been used, read, or been scanned and that there may be a breach in sterility especially if the medicament delivery device is a single use/disposable medicament delivery device, and/or that the medicament has expired.

A user may have various physiological disabilities or impairments, such as dementia or blindness, and may not remember whether he/she has taken the medication or may not see whether the medicament delivery device is the correct one if the user takes different types of medications. The risk of accidental medicament administration, administration of incorrect medicament, and/or administration of expired medicament may thus be reduced.

According to one embodiment the gripping device is configured to grip the portion of the housing of the medicament delivery device when received in the opening.

The actuation of the gripping devices involves moving the gripping device towards a central longitudinal axis of the opening so that it grips the portion of the housing.

According to one embodiment the gripping device comprises first gripping members configured to grip the protective cap and second gripping members configured to grip the portion of the housing, wherein the motor system is configured to drive the first and second gripping members so that at least one of the first and second gripping members are able to move transversally and longitudinally in relation to a longitudinal axis of the opening.

The gripping device may thereby be able to remove the cap from the housing of the medicament delivery device. This may further facilitate protective cap removal for patients with impaired finger skills.

Transversal and/or longitudinal movement of the first and/or second gripping members may for example be provided by means gears and or a linkage system. The gripping device may thus for example comprise gears and/or a linkage system.

According to one embodiment the control unit is configured to control the motor system to actuate the gripping device so that in a first step the first gripping members and the second griping members move transversally inwards, towards the central longitudinal axis, in the opening for gripping a respective one of the protective cap and the housing, and so that in a second step the first gripping members and/or the second griping members move longitudinally for removing the protective cap from the housing.

According to one embodiment the control unit is configured to control the motor system to actuate the gripping device so that in a third step the second gripping members move transversally outwards in the opening, away from the central longitudinal axis, for releasing the portion of the housing such that the medicament delivery device can be removed from the opening.

According to one embodiment the opening is configured to receive a protective cap having any shape.

One embodiment comprises a power source configured to power the motor system and the sensor unit.

The power source may for example comprise one or more rechargeable batteries or one or more interchangeable batteries.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows a block diagram of an example of an electromechanical cap remover;

FIG. 2 schematically shows a cross-section of an example of an electromechanical cap remover; and

FIG. 3 schematically shows a cross-section of another example of an electromechanical cap remover.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

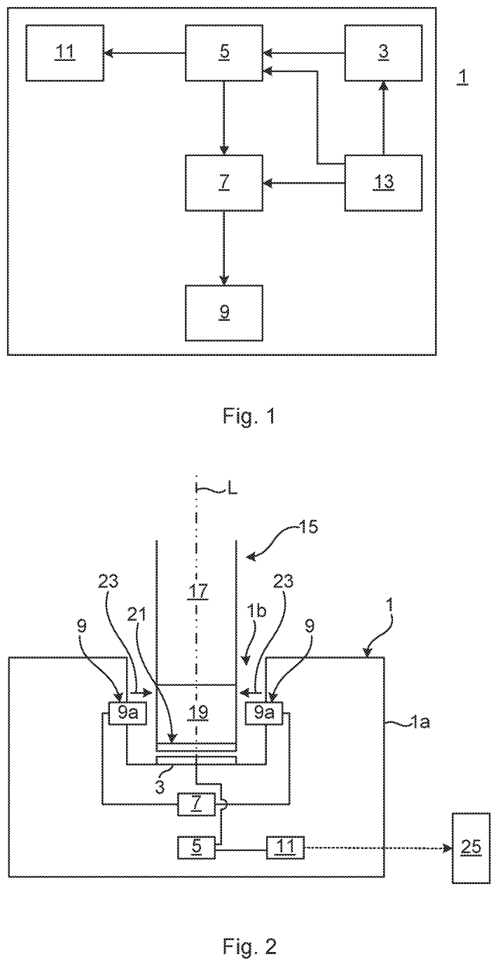

FIG. 1 shows a block diagram of an example of an electromechanical cap remover 1. The electromechanical cap remover 1 is configured to remove a protective cap from a medicament delivery device. The medicament delivery device may for example be an injector, auto-injector, an inhaler or an eye dispenser.

Medicament delivery devices suitable for use with the electromechanical cap remover 1 have a housing and a protective cap that is removably attached to the housing. The medicament delivery device comprises a medicament container containing a medicament and arranged in the housing, and a delivery member configured to expel medicament from the medicament container. The protective cap is arranged at the front end of the housing to protect the delivery member.

The electromechanical cap remover 1 has a cap remover housing provided with an opening (not shown in FIG. 1) configured to receive the protective cap fitted onto the housing of a medicament delivery device and a portion of the housing of the medicament delivery device adjacent to the protective cap.

The electromechanical cap remover 1 comprises a sensor unit 3, a control unit 5, a motor system 7 and a gripping device 9.

The sensor unit 3 is configured to detect the presence of the protective cap and the portion of the housing when the medicament delivery device is placed in the opening. The sensor unit 3 may be arranged in the opening. The sensor unit 3 may for example comprise an optical sensor or any other sensor suitable for detecting the presence of a protective cap and housing.

The control unit 5 is configured to receive detection information from the sensor unit 3 that the senor unit 3 has detected the presence of the protective cap and the portion of the housing in the opening. The control unit 5 is configured to control the motor system 7 based on the detection information.

The motor system 7 may comprise one or more motors. The motor system 7 is configured to actuate the gripping device 9. The control unit 5 is configured to control the motor system 7 to actuate the gripping device 9 in response to receiving detection information from the sensor unit 3 that it has detected the presence of the protective cap and the portion of the housing in the opening. This actuation involves moving the gripping device 9 such that it grips the protective cap.

The number of motors of the motor system 7 depend on the number of degrees of freedom with which the gripping device 9 is configured to operate. For example, the gripping device 9 may be configured to move transversally relative to the central longitudinal axis of the opening and longitudinally, i.e. parallel with the central longitudinal axis. A respective motor may be required for operating the gripping device 9 to move transversally and longitudinally.

The sensor unit 3 may be configured to detect that the cap is maintained in the opening and that the portion of the housing is removed from the opening.

The sensor unit 3 may be configured to detect that the protective cap is removed from the medicament delivery device, i.e. from the housing.

The protective cap may be provided with a label with a barcode, a colour code, a QR code and/or an RFID tag or chip Alternatively and/or additionally, the housing of the medicament delivery device may be provided with a label with a barcode, a QR code and/or an RFID tag or chip. The barcode, QR code and/or RFID tag or chip may comprise data or information related to the medicament delivery device and/or to the medicament contained in the medicament container. For example, the data or information may comprise a unique identifier identifying the medicament delivery device and/or the medicament. The label may according to one example be a non-reversible temperature label configured to indicate whether a predetermined temperature such as upper threshold temperature for the medicament has been reached. The sensor unit 3 may be configured to read the non-reversible temperature label.

The sensor unit 3 may comprise a reader or sensor such as a barcode reader, a QR code reader, a colour sensor and/or an RFID tag or chip reader. The sensor unit 3 may be configured to read the barcode, the QR code, the colour code and/or the RFID tag or chip to obtain the data or information related to the medicament delivery device and/or to the medicament. The sensor unit 3 may comprise a light source. The sensor unit 3 may for example comprise a Light Emitting Diode (LED) light source and a colour sensor such as Red Green Blue (RGB) sensor configured to read colours, in particular colour codes.

The sensor unit 3 may comprise any combination of the above-mentioned readers/sensors.

The electromechanical cap remover 1 may comprise a communication module 11. The communication module 11 may be configured for wireless and/or wired communication. The communication module 11 is configured to communicate with an external device.

The communication module 11 may for example be configured to communicate using Bluetooth®, or Light fidelity (Li-Fi). The communication module 11 may be configured to communicate using local area network-based wireless communication, or cellular network-based wireless communication. The local area network-based communication may for example support any standard in the IEEE 802.11 protocol suite.

The communication module 11 may be configured to receive the data or information obtained by the sensor unit 3. The communication module 11 may be configured to send the data or information to the external device wirelessly or by wired communication. For example, the sensor unit 3 may be configured to send the data or information to the control unit 5, which in turn may be configured to communicate with the communication module 11. Alternatively, the sensor unit 3 may be configured to send the data or information to the communication module 11 directly.

The electromechanical cap remover 1 may have a power source 13. The power source 13 is configured to power the motor system 7. The power source 13 may be configured to power the sensor unit 3. The power source 13 may be configured to power the control unit 5. The power source 13 may be configured to power the communication module 11.

According to one variation the electromechanical cap remover 1 may be configured to be plugged into a power outlet for powering the electromechanical cap remover 1.

FIG. 2 schematically shows an example of an electromechanical cap remover 1 in cross-section. The electromechanical cap remover 1 has a cap remover housing 1a. The cap remover housing 1a is provided with an opening 1b configured to receive a portion of a medicament delivery device 15, including a portion of a housing 17 and a protective cap 19 attached to the housing 17.

In the present example, the protective cap 19 is provided with a label 21 such as a QR label, a label with a colour code, and/or a barcode label. The label 21 could alternatively be an RFID tag or chip. The QR label, label with colour code, barcode label and/or RFID tag or chip is provided with data or information about the medicament delivery device 15 and/or the medicament contained in the medicament delivery device 15. The label 21 may according to one example include a non-reversible temperature label that can be read by the sensor unit.

The sensor unit 3 is arranged in the opening 3. According to the present example the sensor unit 3 is arranged at the bottom of the opening 1b. The sensor unit 3 is configured to read the label 21 when the protective cap 19 is arranged in the opening 3. The sensor unit 3 is configured to detect the presence of the protective cap 19 by detecting the label 21.

The sensor unit 3 may be configured to detect a portion of the housing 17 in the opening 1b. The sensor unit 3 may for this purpose be provided with an additional sensor.

The gripping device 9 may comprise first gripping members 9a that are configured to be moved transversally by the motor system 7, as shown by arrows 23. The first gripping members 9a may be arranged opposite to each other in the opening 1b. The first gripping members 9a may be configured to move towards the central longitudinal axis L of the opening 1b and of the medicament delivery device 15 when the medicament delivery device 15 is placed in the opening 1b. The first gripping members 9a may be configured to move towards each other for gripping the protective cap 19.

The gripping device 9 may comprise gears or a linkage system for moving or driving the first gripping members 9a transversally.

The operation of the electromechanical cap remover 1 depicted in FIG. 2 will now be described.

A user who wishes to use the medicament delivery device 15 places the medicament delivery device 15 with the protective cap 19 attached to the housing 17 into the opening 1b. The protective cap 19 is oriented downwards as shown in FIG. 2.

The sensor unit 3 detects the presence of the protective cap 19. The sensor unit 3 provides detection information that the protective cap 19 has been detected in the opening 1b to the control unit 5.

The sensor unit 3 also read the data or information contained in the label 21. The sensor unit 3 provides the data or information to the communication module 11. The communication module 11 sends the data or information wirelessly or by wire to an external device 25 to register the data or information.

The control unit 5 controls the motor system 7 to actuate the gripping device 9 in response to receiving the detection information. The control unit 5 controls the motor system 7 to move the first gripping members 9a towards the central longitudinal axis L and thus towards the protective cap 19. The first gripping members 9a will thus eventually grip the protective cap 19. The first gripping members 9a only grip the protective cap 19. The first gripping members 9a do not grip the housing 17.

The gripping of the protective cap 19 may be indicated by the electromechanical cap remover 1 for example by means of a visual or aural indicator. This may provide feedback for the user that the medicament delivery device 15 can be removed from the opening 1b.

After the protective cap 19 has been gripped by the gripping device 9, the user may remove the medicament delivery device 15 from the opening 1b. The protective cap 19 is temporarily maintained in the opening 1b. The protective cap 19 may be removed from the opening 1b by the user.

Once the user has removed the medicament delivery device 15 from the opening 1b, the user may commence with medicament administration.

The sensor unit 3 may detect that the protective cap 19 has been removed from the housing 17. The communication module 11 may be configured to send information to the external device 25 that the protective cap 19 has been removed.

All the communication by the communication module 11 may for example be performed after the gripping device 9 has gripped the protective cap 19 and the user has removed the protective cap 19 from the housing 17 by removing the medicament delivery device 15 from the opening 1b. Alternatively, communication by the communication module 11 may be provided as the sensor unit 5 detects the presence of the protective cap 19 and as the housing 17 is removed from the opening 1b.

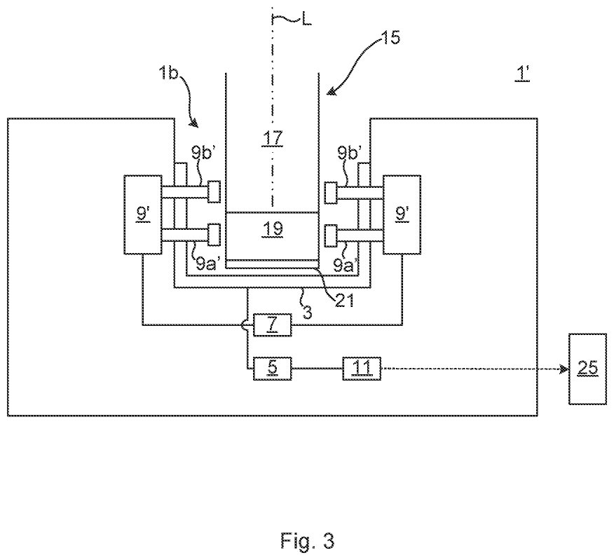

FIG. 3 shows another example of an electromechanical cap remover 1′. The electromechanical cap remover 1′ is similar to the electromechanical cap remover 1 shown in FIG. 2. The gripping device 9′ is in this example however provided with first gripping members 9a′ and second gripping members 9b′.

The first gripping members 9a′ may be arranged opposite to each other. The first gripping members 9a′ may be arranged in level with each other. The first gripping members 9a′ may be configured to grip only the protective cap 19 when arranged in the opening 1b.

The second gripping members 9b may be arranged opposite to each other. The second gripping members 9b′ may be arranged in level with each other. The second gripping members 9b′ may be arranged axially offset from the first gripping members 9a′ along the central longitudinal axis L of the opening 1b. The second gripping members 9b′ are configured to grip a portion of the housing 17. The second gripping members 9b′ may be configured to only grip a portion of the housing 17.

In the example in FIG. 3 the first gripping members 9a′ are configured to be moved transversally relative to the central longitudinal axis L by the motor system 7. The second gripping members 9b′ are configured to be moved transversally and longitudinally relative to the central longitudinal axis L. It could alternatively be the other way around, or both the first gripping members and the second gripping members could be configured to be moveable transversally and longitudinally.

The operation of the electromechanical cap remover 1′ will now be described. When a user desires to use the medicament delivery device 15, the medicament delivery device 15 with the protective cap 19 is placed in the opening 1b with the protective cap 19 facing down.

The sensor unit 3 reads the data or information on the label 21 provided on the protective cap 19. The data or information read by the sensor unit 3 is provided to the communication module 11, which transmits the data or information to the external device 25.

The control unit 5 receives detection information from the sensor unit 3 that the protective cap 19 and a portion of the housing 17 has been detected. The detection information may for example be based on the data or information read by the sensor unit 3. This applies to any example disclosed herein.

The control unit 5 controls the motor system 7 to actuate the gripping device 1 in response to receiving the detection information.

According to the example, the control unit 5 controls the motor system 7 such that the motor system 7 first actuates the first gripping members 9a′ and the second gripping members 9b′ to move transversally towards the central longitudinal axis L. The first gripping members 9a′ grip the protective cap 19 and the second gripping members 9b′ grip the housing 17.

The control unit 5 then controls the motor system 7 such that the second gripping members 9ab′ move longitudinally upwards, away from the bottom of the opening 1b. The first gripping members 9a′ remain axially fixed and maintain their grip of the protective cap 19. The housing 17 is thereby removed from the protective cap 19. This further facilitates removal of the protective cap 19 for users with impaired finger skills.

According to one variation, the first gripping members are instead moved downwards while the second gripping members are kept axially fixed, to remove the protective cap. Alternatively, the first gripping members are moved downwards and the second gripping members are moved upwards to separate the protective cap from the housing.

When the second gripping members 9b′ and/or the first gripping members 9a′ have moved longitudinally to remove the cap 19, the control unit 5 is configured to control the motor system 7 to actuate the second gripping members 9b′ transversally away from the central longitudinal axis L to release the housing 17. The user can then remove the medicament delivery device 15 from the opening 1b without its protective cap 19. The medicament delivery device 15 is then ready to be used for medicament administration.

The transversal and longitudinal movement of the first/second gripping members may for example be implemented by means of a plurality of gears and/or a linkage system.

According to any example disclosed herein, the communication module may be configured to receive feedback data from the external device, the feedback data being based on the data or information sent by the communication module to the external device. The control unit may be configured to control the motor system to actuate the gripping device based on the detection information and on the feedback data. The feedback data may comprise instructions that prevents the control unit from controlling the motor system to actuate the gripping device to grip the protective cap. These instructions may be based on the data or information read from e.g. the barcode, QR code, colour code and/or an RFID tag or chip provided on the protective cap and/or housing of the medicament delivery device. The external device may for example be configured to process the data or information and determine that the unique identifier has been detected before and the medicament delivery device is a single use or disposable device, or that the best before date of the medicament has expired. In this case the feedback data comprises instructions that prevents the control unit from controlling the gripping device to grip the protective cap. In case the data or information indicates that everything is in order, the feedback data comprises instructions that the control unit is to control the motor system to actuate the gripping device to grip the protective cap as their presence has been detected, which is known from the detection information. That everything is in order may for example mean that it is determined that the medicament delivery device has not been used before and that the medicament has not yet expired.

The electromechanical cap remover may comprise an indicator configured to provide feedback to a user based on the feedback data. The control unit may be configured to control the indicator based on the content of the feedback data. For example, in case the feedback data comprises instructions that prevents the control unit from controlling the motor system to actuate the gripping device to grip the protective cap, the control unit may be configured to control the indicator to provide feedback about why the electromechanical cap remover will not remove the protective cap from the medicament delivery device. Such indications may for example be “medicament expired” or “medicament delivery device has previously been used” or something similar, indicated visually or aurally.

The control unit may comprise processing circuitry for processing the detection information and the feedback data, and for controlling the motor system and optionally the indicator. The control unit may comprise a storage medium comprising computer code which when executed by the processing circuitry causes the control unit to control the motor system based on the detection information and the feedback data as disclosed herein.

The processing circuitry may for example use any combination of one or more of a suitable central processing unit (CPU), multiprocessor, microcontroller, digital signal processor (DSP), application specific integrated circuit (ASIC), field programmable gate arrays (FPGA) etc., capable of executing any herein disclosed operations concerning motor system control and optionally indicator control.

The storage medium may for example be embodied as a memory, such as a random access memory (RAM), a read-only memory (ROM), an erasable programmable read-only memory (EPROM), or an electrically erasable programmable read-only memory (EEPROM) and more particularly as a non-volatile storage medium of a device in an external memory such as a USB (Universal Serial Bus) memory or a Flash memory, such as a compact Flash memory.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15: Electromechanical cap remover for removing a protective cap from a medicament delivery device, the electromechanical cap remover comprising:
a cap remover housing having an opening configured to receive the protective cap removably attached to the medicament delivery device,
a motor system,
a gripping device configured to be actuated by the motor system and to grip the protective cap when received in the opening,
a sensor unit configured to detect that the protective cap and a portion of a housing of the medicament delivery device is placed in the opening,
a control unit configured to receive detection information from the sensor unit that the protective cap and the portion of the housing have been detected,
a communication module configured to receive the detection information from the sensor unit and to send it to an external device, wherein the communication module is further configured to receive feedback data from the external device,
wherein the control unit is configured to control the motor system to actuate the gripping device to grip the protective cap based on the detection information and/or based on the feedback data.

16: The electromechanical cap remover according to claim 15, wherein the sensor unit is configured to detect that the cap is maintained in the opening and that the portion of the housing is removed from the opening.

17: The electromechanical cap remover according to claim 15, wherein the sensor unit is configured to detect that the protective cap is removed from the medicament delivery device.

18: The electromechanical cap remover according to claim 15, wherein the sensor unit is configured to read data or information from the protective cap and/or from any component of the medicament delivery device.

19: The electromechanical cap remover according to claim 18, wherein the sensor unit is configured to read a barcode, QR code, colour code and/or an RFID tag or chip to obtain the data or the information.

20: The electromechanical cap remover according to claim 18, wherein the communication module is configured to receive the data or the information from the sensor unit and to send it to the external device.

21: The electromechanical cap remover according to claim 20, wherein the data or the information comprises data or information related to the medicament delivery device and/or to the medicament.

22: The electromechanical cap remover as claimed in claim 20, wherein the communication module is configured to receive feedback data from the external device, which feedback data is based on the data or information sent to the external device, wherein the control unit is configured to control the motor system to actuate the gripping device further based on the feedback data.

23: The electromechanical cap remover according to claim 15, wherein the gripping device is configured to grip the portion of the housing of the medicament delivery device when received in the opening.

24: The electromechanical cap remover according to claim 23, wherein the gripping device comprises first gripping members configured to grip the protective cap and second gripping members configured to grip the portion of the housing, wherein the motor system is configured to drive the first and second gripping members so that at least one of the first and second gripping members are able to move transversally and longitudinally in relation to a longitudinal axis (L) of the opening.

25: The electromechanical cap remover according to claim 24, wherein the control unit is configured to control the motor system to actuate the gripping device so that in a first step the first gripping members and the second griping members move transversally inwards in the opening, towards the central longitudinal axis (L), for gripping a respective one of the protective cap and the housing, and so that in a second step the first gripping members and/or the second griping members move longitudinally for removing the protective cap from the housing.

26: The electromechanical cap remover according to claim 25, wherein the control unit is configured to control the motor system to actuate the gripping device so that in a third step the second gripping members move transversally outwards in the opening, away from the central longitudinal axis (L), for releasing the portion of the housing such that the medicament delivery device can be removed from the opening.

27: The electromechanical cap remover according to claim 15, wherein the opening is configured to receive a protective cap having any shape.

28: The electromechanical cap remover according to claim 15, comprising a power source configured to power the motor system and the sensor unit.

29: Electromechanical cap remover for removing a protective cap from a medicament delivery device, the electromechanical cap remover comprising:
a cap remover housing having an opening configured to receive the protective cap when removably attached to the medicament delivery device;
a motor system;
a gripping device operatively connected to the motor system;
a sensor unit that automatically detects when the protective cap attached the medicament delivery device is placed within the opening;
a control unit that receives detection information from the sensor unit that the protective cap attached to the medicament delivery device; and
a communication module that receives the detection information from the sensor unit and sends the detection information to an external device, where the communication module is configured to receive feedback data from the external device,
wherein the gripping device comprises first gripping members that grip the protective cap automatically when the protective cap is placed within the opening and where second gripping members grip a portion of an outer housing of the medicament delivery device,
wherein the motor system is configured to drive the first and second gripping members so that at least one of the first and second gripping members are able to move transversally and longitudinally in relation to a longitudinal axis (L) of the opening.

30: The electromechanical cap remover of claim 29, wherein the control unit controls the motor system to actuate the first and second gripping members based on the detection information or based on the feedback data.

31: The electromechanical cap remover of claim 30, wherein the sensor unit reads data or information from a barcode, QR code, colour code or an RFID tag or chip located on or in the protective cap.

32: The electromechanical cap remover of claim 31, wherein the sensor unit reads data or information from a barcode, QR code, colour code or an RFID tag or chip located on or in any component of the medicament delivery device.

33: The electromechanical cap remover according to claim 29, further comprising a power source to power the motor system and the sensor unit.
